(19) United States
(12) Patent Application Publication
(10) Pub. No.: US 2023/0346224 A1
(43) Pub. Date: Nov. 2, 2023

(54) A DISPOSABLE COVER FOR COVERING A HEAD OF AN OPTICAL DEVICE

(71) Applicants: Anupam LAVANIA, Satellite, Ahmedabad, Gujarat (IN); Shilpa MALIK, Satellite, Ahmedabad, Gujarat (IN)

(72) Inventors: Anupam LAVANIA, Satellite, Ahmedabad, Gujarat (IN); Shilpa MALIK, Satellite, Ahmedabad, Gujarat (IN)

(21) Appl. No.: 17/998,645

(22) PCT Filed: May 11, 2021
(86) PCT No.: PCT/IB2021/053979

§ 371 Date: Nov. 11, 2022

(30) Foreign Application Priority Data
May 12, 2020 (IN) 202021020058

(51) Int. Cl. A61B 5/00

(52) U.S. Cl. CPC A61B 5/0077; A61B 2562/247

(57) ABSTRACT

The present disclosure discloses a disposable cover (4) for covering a head (8) of an optical device (1) and contacting a subject for examination. The cover includes a body (21), defining a first surface (19) and a second surface (20). The body (21) is removably connectable to the head through the first surface and the second surface, where one or more apertures (6) are defined in the first surface (20). A flexible cap (7) is disposed of in each of the one or more apertures of the body, where at least a portion of the flexible cap is made of a substantially transparent material. The flexible cap is structured to accommodate an optical probe (3) extending from the head of the optical device, such that the optical probe extends and retract relative to the body based on position of the optical device relative to the subject.

### TECHNICAL FIELD

Present disclosure, in general, relates to the field of medical devices. Particularly, but not exclusively, the present disclosure relates to an optical device contacting a subject for examination. Further, embodiments of the present disclosure disclose a disposable cover for covering a head of the optical device.

### BACKGROUND OF THE DISCLOSURE

Generally, subjects having medical conditions are monitored by conducting examinations on the subject to identify the medical condition. Also, the examinations may be conducted to quantify the extent of the medical condition suffered by the subject. Such monitoring and examination of the subject may be conducted periodically or at real-time by examination devices [hereafter referred to as device] that may be suitably interfaced with the subject. The device may operate based on principles including, but not limited to, light, sound, pressure, electrical impulses, magnetic impulses, and radiation. The devices may be fixed such that the subject may be moved relative to the device or the device may be mobile relative to the subject for selective examination and monitoring of the subject, based on operational and working principle of said device. Further, the devices may be configured to come in contact with the subject or may operate at a distance from a surface of the subject for monitoring and examination. Typically, the devices that come in contact with the subject employ components to contact at least a portion of surface [such as, skin, muscle, bone, cartilage, and the like] of the subject.

Conventionally, the devices that come in contact with the subject are optical devices employing optical components, where the optical components may be optical lenses, optical mirrors, optical fibers, beam-splitters, polarization optics, diffraction gratings, optical systems, optical windows, prisms, fiber optics, optic sets, custom optic solutions or any other similar component. In some instances the optical components may be periodically replaceable, as such components tend to engage a surface of the subject. The Performance of the device is dependent on cleanliness of the optical components in the device. Any contamination on an end face of the optical components may cause failure of the device. The optical components may be contaminated in many ways for example, particulates, salts, oils, blood, sweat, dust, and the like, which may accumulate on the optical components on contact with the surface of the subject. Further, the contamination of the optical components causes scattering, back reflections, instability in the optical beam, signal attenuation or even a system shutdown and impurities on the optic surface may react with the optical beam to damage optical coatings. Generally, the contamination may be maintained to a minimum by returning the optical device back into the corresponding protective case after use or by covering the optical device for protection from the outside environment. However, even by covering the optical device after use, the contamination cannot be mitigated. Moreover, when the optical device is deployed in a medical environment with possibility of direct contact with an infected surface of the subject, then an external interface needs to be inserted in order to restrict communication of any infectious organisms, hence arresting the spread of communicable diseases. This external interface needs to be sterilized free from any contaminants before its reinstallation. Further, direct contact of the optical components with oil, blood, salts, sweat, and other contaminants, present at the infected surface of the subject results in contamination of the optical components. These optical components upon contamination may have to be removed often for cleaning or purging the contaminations. The frequent disassembly and assembly of the components inside the optical device may damage the components and once damaged, the components have to be replaced, which is expensive. Further, the process of cleaning the component surface is extremely exhaustive, as the contamination in a microscopic scale ensuring complete decontamination is time consuming and purging the contaminants is exhaustive.

With advancements in technologies in the medical equipment area, multiple solutions for cleaning such contaminants on the medical equipment have been developed. Rubbing fluids such as alcohol containing agents or gels on the components is one such method. However, upon continuous use of alcohol or alcohol containing agents, the optical device may get damaged. Similarly, organic acidic solutions also act as cleaning agents, however, such acids are toxic or cause damage to the optical device. Additionally, salt deposits on the optical component surfaces are not fully removed by cleaning fluids alone. While cleaning fluids may quickly rinse away the oils, they tend to leave salt remains or residues behind on the optical components in the form of a white or alike residue that is difficult to remove. Further, mechanical action from a wipe or a swab combined with the cleaning fluid may be used on the components to fully eliminate oil and salt left on the optical components. However, wiping action may leave residue on the end face of optical components that eventually causes damage to performance of the optical device. Nonetheless, as mentioned above, cleaning of the optical components is a tedious and time-consuming process. Further, a close inspection of the optical components is critical to identify the residual particles and eliminate these particles. Consequently, a specialized, say 200×or 400×inspection scope may be required for thorough cleaning of the optical components, which further complicates the process.

Furthermore, the optical components are required to be operated within a certain threshold force. Force beyond the defined threshold may lead to damage of the optical components as bare components are more susceptible to damage. Further, excessive force on the surface of the subject may cause discomfort to the subject.

The present disclosure is directed to overcome one or more limitations stated above or any other limitations associated with the conventional mechanisms.

### SUMMARY OF THE DISCLOSURE

One or more shortcomings of the prior art are overcome by a device as claimed and additional advantages are provided through the device as claimed in the present disclosure. Additional features and advantages are realized through the techniques of the present disclosure. Other embodiments and aspects of the disclosure are described in detail herein and are considered a part of the claimed disclosure.

In one non-limiting embodiment of the present disclosure a disposable cover or cap for covering a head of an optical device and contacting a subject for examination is disclosed. The cover includes a body, defining a first surface and a second surface extending along a periphery of the first surface in a direction substantially vertical to the first surface. The body is removably connectable to the head through the first surface and the second surface, where one or more apertures are defined in the first surface. Further, a flexible cap disposed in each of the one or more apertures of the body, where at least a portion of the flexible cap is made of a substantially transparent material. The flexible cap is structured to accommodate an optical probe extending from the head of the optical device, such that the optical probe extends and retract relative to the body based on position of the optical device relative to the subject.

In an embodiment, the body is configured to selectively filter light being either incident or reflected from at least one of the surface of the subject and surrounding of the body onto the head of the optic device.

In an embodiment, the body includes a stepped portion extending from the first surface, where the stepped portion is defined with one or more apertures to accommodate the optical probe.

In an embodiment, the flexible cap is positioned relative to the first surface of the body based on selective accommodation of the optical probes in the one or more apertures.

In an embodiment, the flexible cap is configured to extend away from the first surface on receiving the optical probe to contact the subject

The flexible cap defines a curved profile, when viewed along a cross-section of the flexible cap and the body at a connection portion with the one or more aperture on extension of the flexible cap away from the first surface.

In an embodiment, the flexible cap is configured to retract and flush with the first surface of the body, on contacting the subject for examination. In this condition, The flexible cap defines a substantially triangular profile, when viewed along a cross-section of the flexible cap and the body at the connection portion with the one or more aperture, on retraction of the flexible cap to flush with the first surface.

In an embodiment, the cover includes at least one protrusion defined on the first surface of the body. The at least one protrusion is receivable in a cavity defined in the head of the optical device to operate the optical device. The at least one protrusion is structured to activate a sensor in the head of the optical device on being received in the cavity to operate the optical device.

In an embodiment, the cover includes a locking mechanism defined on at least one of the body and the head of the optical device, where the locking mechanism is configured to lock the head of the optical device to secure the cover. Further, the locking mechanism includes at least one locking tab defined on the second surface of the body and a locking member defined in the head of the optical device. The at least one locking tab is aligned and displaceable relative to the at least one locking member to lock the cover with the head of the optical device.

In an embodiment, the first surface of the cover is provisioned with a plurality of ribs defining air pockets between the cover and the head of the optical device.

In an embodiment, the second surface of the body is provisioned with a seal which is configured to be torn-off to attach the cover on the head of the optical device.

In an embodiment, the first surface and the second surface are defined with a profile complementary to a profile of the head of the optical device.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE ACCOMPANYING DRAWINGS

The novel features and characteristics of the disclosure are set forth in the appended claims. The disclosure itself, however, as well as a preferred mode of use, further objectives, and advantages thereof, will best be understood by reference to the following detailed description of an illustrative embodiment when read in conjunction with the accompanying figures. One or more embodiments are now described, by way of example only, with reference to the accompanying figures wherein like reference numerals represent like elements and in which:

FIG. 1 illustrates a perspective view of the optical device, in accordance with an embodiment of the disclosure.

FIG. 2 illustrates a perspective view of the optical device along with a cover, in accordance with an embodiment of the disclosure.

FIG. 3 illustrates a perspective view of the cover along with optical probes, in accordance with an embodiment of the disclosure.

FIG. 4 illustrates a sectional view of the cover along with the optical probes, in accordance with an embodiment of the disclosure.

FIG. 5 illustrates a front sectional view of the cover, where the optical probe from a head is inserted into the cap of the cover, in accordance with an embodiment of the disclosure.

FIG. 6 illustrates a magnified view of the portion A of the FIG. 5, in accordance with an embodiment of the disclosure.

FIG. 7A illustrates a front sectional view of the cover, when the optical device is completely in contact with a surface and the cap is of hard material, in accordance with an embodiment of the disclosure.

FIG. 7B illustrates a front sectional view of the cover, when the optical device is in contact with the surface and the cap is of soft and flexible material, in accordance with an embodiment of the disclosure.

FIG. 8A illustrates a magnified view of the portion B of the FIG. 7A, in accordance with an embodiment of the disclosure.

FIG. 8B illustrates a magnified view of the portion B of the FIG. 7B, in accordance with an embodiment of the disclosure.

FIG. 9 and FIG. 10 illustrate a magnified view of the cap along with the optical probe and also indicates the particulars of the first method for the choice of material (flexible/collapsible) of the cap, in accordance with an embodiment of the disclosure.

FIG. 11 and FIG. 12 illustrates a magnified view of the cap along with the optical probe and also indicates the particulars of the second method for the choice of material (rigid) of the cap, in accordance with an embodiment of the disclosure.

FIG. 13 illustrates a tip of the cap with a cylindrical and a conical portion, in accordance with an embodiment of the disclosure.

FIG. 14 illustrates a schematic view of the head and the cover when the head is not in contact with the surface, in accordance with an embodiment of the disclosure.

FIG. 15 illustrates a schematic view of the head and the cover when the head is pressed against the surface, in accordance with an embodiment of the disclosure.

FIG. 16 illustrates a rear perspective view of cover defined with at least one locking tab, in accordance with an embodiment of the disclosure.

FIG. 17 illustrates a perspective view of the head defined with locking members, in accordance with an embodiment of the disclosure.

FIG. 18 illustrates a cross-sectional view of a fitment portion of the cover along with the head when viewed with reference to section B-B of FIG. 16.

FIG. 19 illustrates a rear perspective view of the cover, in accordance with an embodiment of the disclosure.

FIG. 20 illustrates a side view showing attachment of the optical device with the cover, in accordance with an embodiment of the disclosure.

FIG. 21 illustrates a rear perspective view of the cover, in accordance with an embodiment of the disclosure.

FIG. 22 illustrates a rear perspective view of the cover with a seal, in accordance with an embodiment of the disclosure.

FIG. 23 illustrates a rear perspective view of the cover and the head with a peeler, in accordance with an embodiment of the disclosure.

The figures depict embodiments of the disclosure for purposes of illustration only. One skilled in the art will readily recognize from the following description that alternative embodiments of the system and method illustrated herein may be employed without departing from the principles of the disclosure described herein.

### DETAILED DESCRIPTION

The foregoing has broadly outlined the features and technical advantages of the present disclosure in order that the detailed description of the disclosure that follows may be better understood. Additional features and advantages of the disclosure will be described hereinafter which form the subject of the claims of the disclosure. It should be appreciated by those skilled in the art that the conception and specific embodiments disclosed may be readily utilized as a basis for modifying other devices, mechanisms, systems, assemblies, methods, and processes for carrying out the same purposes of the present disclosure. It should also be realized by those skilled in the art that such equivalent constructions do not depart from the scope of the disclosure as set forth in the appended claims. The novel features which are believed to be characteristics of the disclosure to its system, together with further objects and advantages will be better understood from the following description when considered in connection with the accompanying figures. It is to be expressly understood, however, that each of the figures is provided for the purpose of illustration and description only and is not intended as a definition of the limits of the present disclosure.

The terms “comprises”, “comprising”, or any other variations thereof, are intended to cover a non-exclusive inclusions, such that a mechanism, a system, or a device that comprises a list of components or steps does not include only those components or steps but may include other components or steps not expressly listed or inherent to such setup or device or method. In other words, one or more elements in a system or apparatus preceded by “comprises . . . a” does not, without more constraints, preclude the existence of other elements or additional elements in the system or apparatus.

In accordance with various embodiments of the present disclosure, a disposable cover for covering a head of an optical device being configured to contact a subject for examination is disclosed. The cover of the present disclosure may be used as fitment or attachment to the optical device and may be disposed after use to maintain sterility of the optical device. The cover according to various embodiments include a body, defining a first surface and a second surface extending along a periphery of the first surface in a direction substantially vertical to the first surface. The body may be removably connectable to the head through the first surface and the second surface, where one or more apertures are defined in the first surface. Further, a flexible cap is disposed in each of the one or more apertures of the body, where at least a portion of the flexible cap may be made of a substantially transparent material. The flexible cap may be structured to accommodate an optical probe extending from the head of the optical device, such that the optical probe extends and retract relative to the body based on position of the optical device relative to the subject. The disposable cover protects the head of the optical device and the optical probes from contamination during examination and prevents damage.

Reference will now be made to the exemplary embodiments of the disclosure, as illustrated in the accompanying drawings. Wherever possible, same numerals have been used to refer to the same or like parts. The following paragraphs describe the present disclosure with reference to FIGS. 1 and 23. It is to be noted that the cover may be employed in any device employed to examine the subject which operates on the principles of, including but not limited to optics, sonics, radiation and the like.

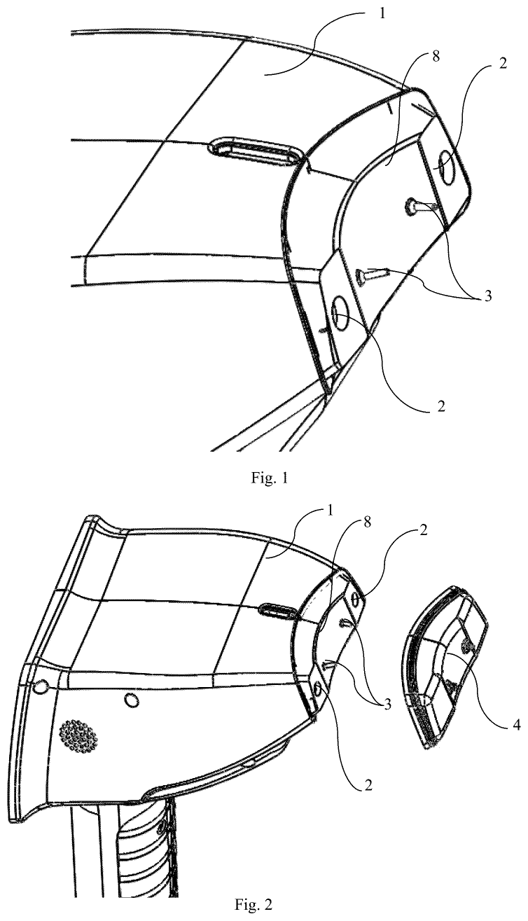

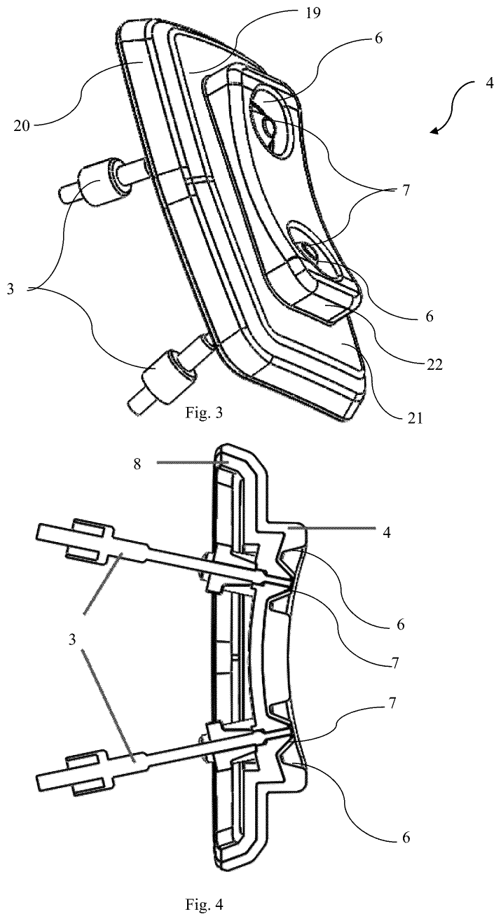

FIGS. 1 and 2 are an exemplary embodiment of the present disclosure which illustrate perspective views of an optical device (1). The optical device (1) includes a sensor head (8) [hereafter referred to as head] in which at least one sensor may be provided. The sensor may be optical probes (3) that may be provided at a substantially central portion of the head (8) and also at any location of the head (8) other than the central portion of the head (8). The optical probes (3) may be customized fiber optic probes integrated with one or more components like lenses. The optical probes (3) may be configured to examine a subject, on contact with a surface of the subject. In an embodiment, the optical probes (3) may be including but not limited to optical lenses, optical mirrors, optical fibers, beam-splitters, polarization optics, diffraction gratings, optical systems, optical windows, prisms, fiber optics, optic sets, custom optic solutions and any other similar component capable of receiving/capturing and transmitting optical signals. Further, the head (8) may be defined with a plurality of cavities, which may be provisioned with a sensor system housed inside the cavity (2). The sensor system may be employed to activate or operate the optical device (1). In an embodiment, the sensor system may be operable mechanically, electronically, electrically, hydraulically, pneumatically or any other combinations thereof. Further, as seen in FIG. 2, the head (8) of the optical device (1) may be adapted to receive a disposable cover (4) or a disposable cap.

In some embodiment, a portion of the head (8) comprising the optical probes (3) is defined with a curved profile, such that the profile matches the portion of the subject to be examined.

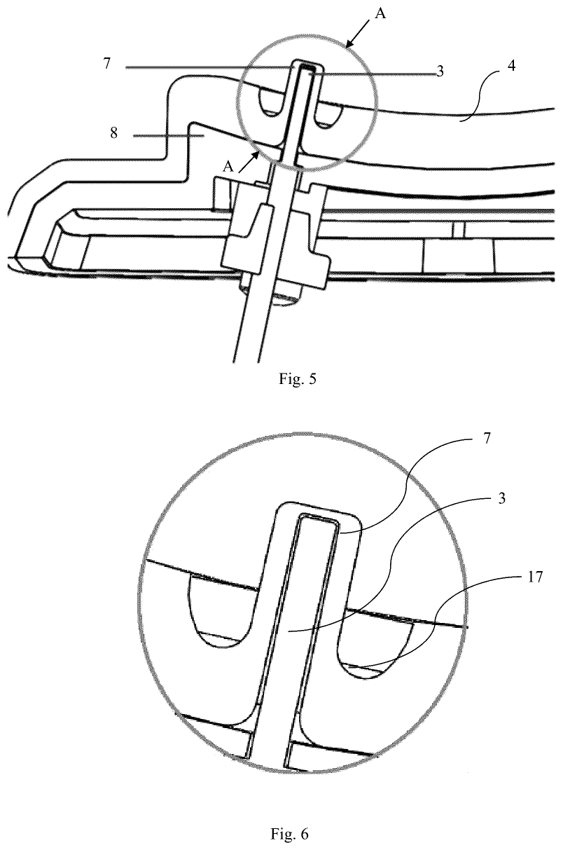

FIGS. 3 and 4 illustrate a perspective view and a side sectional view of the disposable cover (4) or the cap [hereafter referred to as “the cover (4)”] along with the optical probes (3) upon being fixed on the head (8) of the optical device (1). The cover (4) includes a body (21) that is defined with a first surface (19) and a second surface (20), where the second surface (20) may extend along a periphery of the first surface (19). The body (21) of the cover (4) may be adapted to be removably connected to the head (8) through the first surface (19) and the second surface (20). The cover (4) may be connected to the head (8) of the optical device (1), such that the cover (4) enclose the surfaces of the head (8) adapted to engage with the subject. In an embodiment, the first surface (19) and the second surface (20) are defined with a profile complementary to a profile of the head (8) of the optical device (1) to sealingly connect the cover (4) on the head (8) of the optical device (1). The second surface (20) may extend in a direction substantially vertical to the first surface (19). It can be construed that the term “substantially vertical” resembles a reference plane and/or axis to define structural configuration between the first surface (19) and the second surface (20) of the body (21). Such reference with respect to the vertical may be defined as angular orientation between the first surface (19) and the second surface (20) of the body (21), where such angular orientation may be in the range of 10 to 170 degrees relative to such vertical. In an exemplary embodiment illustrated in FIGS. 3 and 4, the first surface (19) and the second surface (20) may be defined with the angular orientation ranging from 60-90 degrees, which may be varied based on parameters including but not limited to rigidity of the body (21), configuration of the cover (4), profile of the head (8) of the optical device (1), and any other parameter affecting securement of the cover (4) on the head (8) of the optical device (1).

The body (21) may be further defined with one or more apertures (6) in the first surface (19), as best seen in FIG. 3. In an exemplary embodiment, the body (21) includes a stepped portion (22) extending from and above the first surface (19), away from the head (8) of the optical device (1). The stepped portion (22) may be defined with the one or more apertures (6) to accommodate the optical probe (3) extending from the head (8) of the optical device (1). The one or more apertures (6) may include a flexible cap (7) disposed in each of the one or more apertures (6). In an embodiment, the flexible cap (7) may be fixed within each of the one more apertures (6) or may be formed as an integral member of the body (21). Further, the flexible cap (7) may be structured to accommodate the optical probe (3) extending from the head (8) of the optical device (1), such that the optical probe (3) extends and retracts relative to the body (21) based on position of the optical device (1) relative to the subject. Furthermore, the flexible cap (7) may be made of a flexible material that allows the flexible cap (7) to move along with the optical probes (3) in the longitudinal direction or along the longitudinal axis of the flexible cap (7). The flexible cap (7) may be made of materials having different flexibility such as, but not limited to, silicon, nylon, PVC, PMC, metals, alloys, and any other material capable of being shaped to receive the optical probes (3). Additionally, the flexibility of the flexible cap (7) may be based on the material employed for manufacturing the flexible cap (7) which may vary from a flexible material and a rigid material exhibiting rigid properties. Flexibility of the flexible cap (7) to displace (i.e., selective deformation of the flexible cap (7) by extension and retraction) along with the optical probe (3) may be based on material selection or the design, such as manufacturing the flexible cap (7) with an elastic material or collapsible rings that may allow the optical probe (3) to displace back and forth.

In an embodiment, the flexible cap is positioned relative to the first surface (19) of the body (21) based on selective accommodation of the optical probes (3) in the one or more apertures (6). For example, the flexible cap (7) may be disposed in the one or more apertures (6) such that the flexible cap (7) is positioned away from the first surface (19) or positioned flush with the first surface (19) of the body (21), when the optical probes (3) are not accommodated inside the one or more apertures (6). The flexible cap (7) extending away from the first surface (19) of the body (21) may be retracted into the one or more apertures (6) upon contacting the surface of the subject. In another embodiment, the flexible cap (7) may be positioned within the one or more apertures (6) upon receiving the optical probes (3), and the flexible cap (3) may be configured to allow examination of the subject by the optical probes (3) from within the one or more apertures (6) i.e. the flexible cap may define a gap between the optical probe (3) and the surface of the subject for examination.

In an embodiment, the body (21) may be configured to selectively filter light being either incident or reflected from at least one of the surface of the subject and surrounding of the body (21) onto the head (8) of the optical device (1). The body (21) may restrict light such that a dark room effect may be created proximal to the optical probes (3). The body (21) may be made of opaque material and at least a portion of the flexible cap (7) may be made of a substantially transparent material. In another embodiment, the body (21) may be made of substantially transparent material. It can be construed that the term “substantially transparent” defines a surface property of the material. For example, the flexible cap (7) may be made of a material having transparency that may be in a range between 10%-100% to allow the required amount of light to travel through the flexible cap (7). The transparency of the flexible cap (7) allows optical signals to be transmitted and/or received by the optical probes (3). In the exemplary embodiment, as seen in FIGS. 3 and 4, the flexible cap (7) of the cover (4) may be configured to reside on top of the optical probes (3) extending from the head (8) of the optical device (1), which enables the portion of the flexible cap (7) having the transparent region to be defined on top/tip of the optical probes (3). Further, as the body (21) may be made of opaque material, regions of the optical head (8) other than the optical probes (3), are covered by opaque portion of the body (21), which may create a dark chamber for portion of the flexible cap (7) defined with substantially transparent material. The dark chamber around the optical probes (3) created by the cover (4) may be configured to control/restrict entry of ambient light from the surrounding. In an embodiment, the flexible cap (7) may be constructed from a material that allows near infra-red (NIR) light to pass through and may be a filter to the ambient light. Additionally, the color of the region around the surface of the one or more apertures (6) and at a bottom portion of the flexible cap (7) may be black so as to control light.

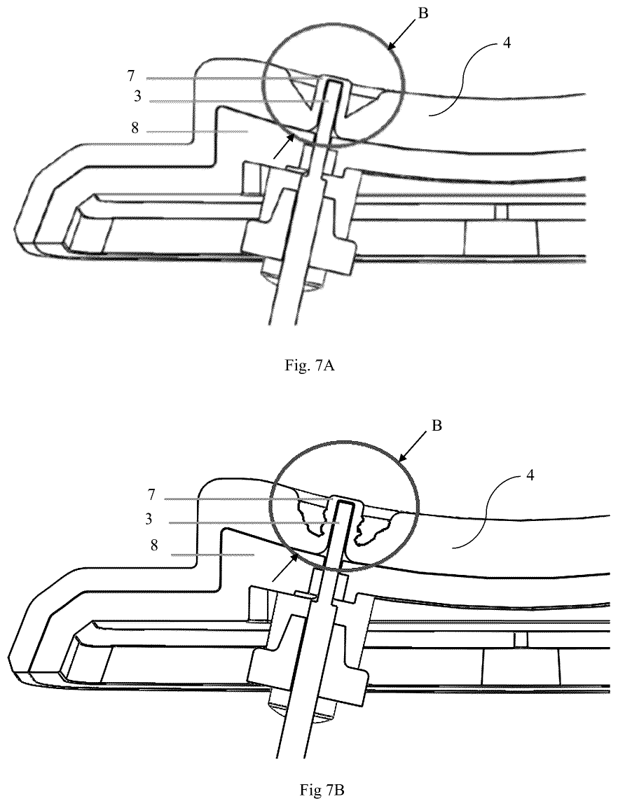

FIG. 5 illustrates a side cross-sectional view of the cover (4), upon the optical probe (3) from the head (8) being inserted into the flexible cap (7) of the cover (4) and FIG. 6 illustrates a magnified view of the section A-A in FIG. 5. Upon connecting the cover (4) on the head (8), the flexible cap (7) of the cover (4) may surround or house a tip portion of the optical probe (3) as seen from the section A-A. in FIG. 6. Further, FIG. 5 depicts an embodiment when the optical device (1) is not brought in contact with any surface. Under such circumstances, pressure is not exerted on the optical probes (3) and the optical probes (3) may have a tendency to extend away from the first surface (19) and remain out of the body (21) of the cover (4) (away from the device) as the optical probes (3) may be spring loaded. Further, the flexible cap (7) surrounding the optical probes (3) also remains projected outwardly away from the device. The base portion of the flexible cap (7) at a connection portion (17) with the one or more apertures (6) in the body (21), defines a curved profile when viewed along a cross-section of the flexible cap (7) and the body (21) [as seen in FIGS. 5 and 6] upon pressure not being applied onto the optical device (1). The curved profile provides a cavity that allows the material of the connection portion (17) and the flexible cap (7) to be accumulated when the optical probes (3) and the flexible portion of the flexible cap (7) is displaced inwards towards the optical head (8). Further, the curved profile ensures extension and retraction of the flexible cap (7).

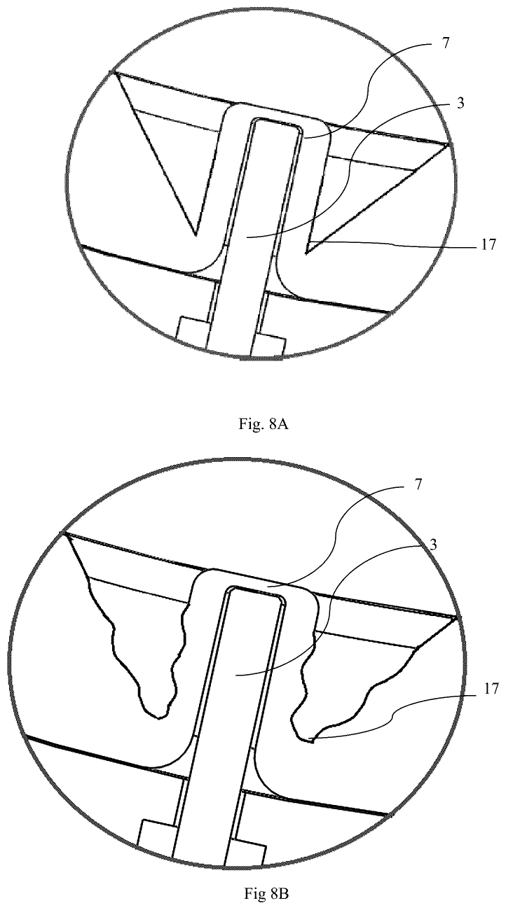

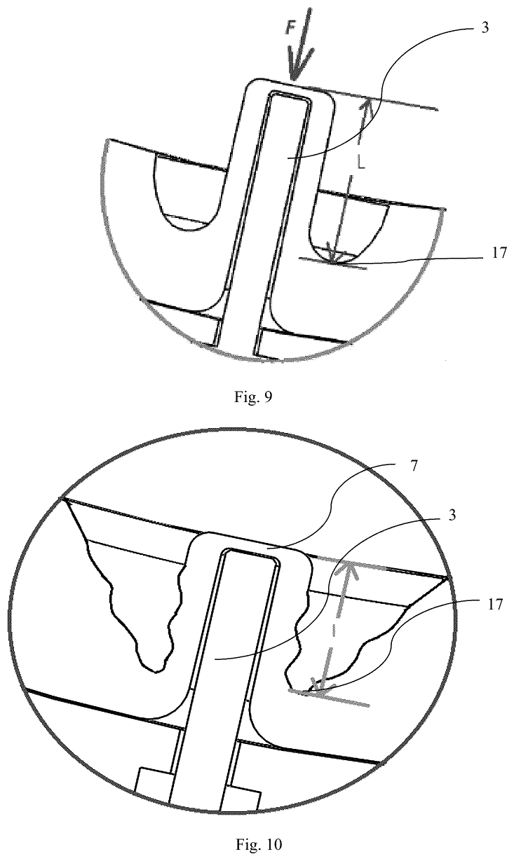

Further, FIG. 7A illustrates a side cross-sectional view of the cover (4), when the device is being in contact with the surface [not shown] of the subject and FIG. 8A illustrates a magnified view of the section B-B of the FIG. 8A. For example, as the optical device (1) is brought in contact with the surface, the optical probes (3) are pushed/displaced in by the virtue of pressure applied by the surface of the subject. Consequently, the flexible cap (7) which may be of a cylindrical shape may be pushed towards the optical device (1) (away from the subject) along with the optical probes (3) due to the pressure. In an exemplary embodiment, the flexible cap (7) may be configured to retract and align flush with the first surface (19) of the body (21) on contacting the subject. Further, on retracting and aligning flush with the first surface (19) as seen from the FIG. 8A, the base portion of the flexible cap (7) at a connection portion (17) with the one or more apertures (6) in the body (21) deforms. The curved profile deforms to a substantially triangular profile, when viewed along the cross-section of the flexible cap (7) [here after referred to as cap (7)] and the body (21). In an embodiment, the flexible cap (7) may or may not be deformed based upon the material it is made up of. The substantially triangular profile may be a profile which may not be limited to a triangle but may also be a profile similar to or resembling a triangle. The substantially triangular profile ensures that the contact between the optical probe (3) and the cap (7) is uniform and there is no air gap left in between. Further, as the optical probes (3) or the cap (7) is pushed inwards i.e., in the fully retracted position the curved portion unfurls to form a linear/straight surface connection with the apertures (6) or the first surface (19) the body. Further, the triangular profile enables the optical probes (3) to have retracting forces, which is enables continuous forces to be transferred onto the subject's surface through the tip of the optical probe (3) and the cap (7).

In an embodiment, the cap (7) may be made of rigid material and the connection portion (17) of the cap (7) may be made of flexible material to be deformed upon movement of the optical probes (3). Further, the cap (7) may be made of a flexible/collapsible material or rigid material. Both the embodiments have different mechanisms which are explained below:

The first mechanism is explained with reference to the FIGS. 7B, 8B and 10 where the cap (7) is made of flexible/collapsible material.

The covering of the optical probes (3) with the cap (7) at all times prevents the direct contact of the optical probes (3) with the surface of the subject and the cap (7) has to be configured to elastically deform under pressure and must regain the original shape when the applied pressure is relieved. This can further be achieved in two ways:

### Example 1

a. The cap (7) is made of elastic material like silicon, nylon and any other elastic or flexible material.

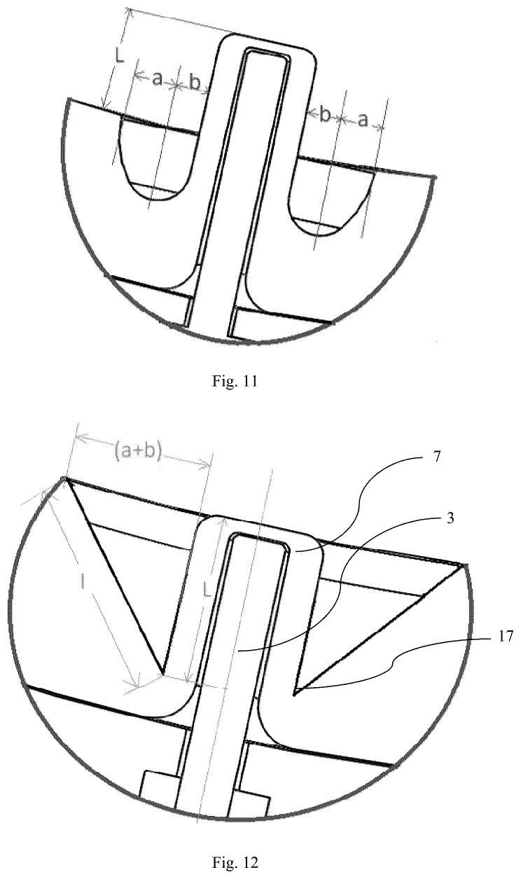

The choice of material of the cap (7) may be made in such a way that the cap (7) is elastic and covers at least a portion of the optical probe (3). For the above disclosed first method, FIGS. 9 and 10 are considered for reference.

Index:
Thickness of a wall of the cap (7), t=t (OD=R, ID=r)
Young's Modulus of the material of the cap (7), =Y
Length of the optical probe (3) from the surface of the optical device (1)=L
Length of the optical probe (3) while fully compressed/retracted=1

Below is the calculation to enable the choice of the material or its Young's modulus(Y):
Y=Stress/Strain
Stress=F/π(R\hat{ }2−r\hat{ }2),
Strain=(L−1)/L=x/L . . . . . . (say)
Now,
(L-1)=x may be fixed by the space available in the optical device (1) directly beneath the optical probe (3), L is determined on the basis of the optical probe (3) depth needed by the optical device (1). Similarly, r=the OD of the optical probe (3) and R is determined on the basis of an inspection site area available for the full contact of the optical probe (a) and the cap (b).
F is the normalized perpendicular force just above the tip of the cap.
b. The walls of the cap (7) are replaced by a resilient member/spring:

The wall of the cap (b) may be replaced by a spring system (e.g. torsional spring, leaf spring, helical spring, and the like) and a spring constant (K) may be determined by the below calculations: F=K X (x), i.e., K=F/(L-1)

### Example 2

The second example may be explained with reference to FIGS. 7A, 8A, 11 and 12 where the cap (7) may be made of hard/nonflexible rigid material.

The cap (7) may be rigid and optimally cover the at least a portion of the optical probe (3) while allowing a predefined wavelength of light to pass from the optical probes (3) to the surface of the subject, upon attaching the cover (4) to the head (8) of the optical device (1). The cap (7) along with the optical probe (3) may be displaced or pushed to retract flush with the first surface (19) of the body (21), while maintaining the rigidity of the cap (7). Further, combination of the cap (7) and the optical probes (3) may be retracted due to flexibility of the base portion of the cap (7) at the connection portion (17) of the cap (7) with the one or more apertures (6) that unfurl to accommodate the combination.

Further, with reference to FIGS. 11 and 12, the radius of the base portion of the cap (7) at the connection portion (17) of the cap (7) with the one or more apertures (6) that covers the area from a bottom of the hard portion of the cap (7) to the first surface (19) of the cover (4)=r (Note: In the fig. Assume a=b=r) and unfurls to be 1=πr. [FIG. 17] Now, in order to determine the r, (using trigonometry) 1\hat{ }2=L\hat{ }2+(2r)\hat{ }2

In an embodiment, the values of the equations provided above may be varied according to the requirement and should not be limited based on the configuration described in the figs.

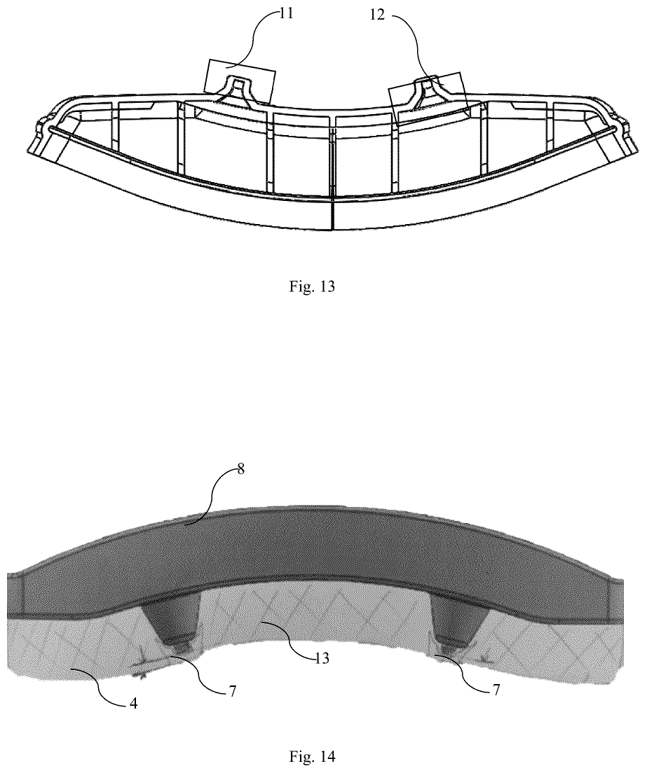

Further, FIG. 13 illustrates an embodiment of the cover (4) where the optical probes (3) are accommodated at a tip of the cover (4) with a cylindrical portion (11) and a conical portion (12). In an embodiment, the tip of the cover (4) may be of two parts. The first part may be the cylindrical portion (11) with a minor draft which may be configured to hold the optical probe (3) tightly. The second part may be a conical portion (12) that may collapse inside the cover (4) upon retraction of the optical probes (3) inside the optical device (1). The conical portion (12) may also be substituted by resilient members including but not limited to springs, collapsible rings, and any other mechanism capable of collapsing. The conical portion (12) may be of any other shape including but not limited to frustum, cubical, cuboidal, cylindrical and the like. In one embodiment, the cylindrical portion (11) may be of hard material. In another embodiment the cylindrical portion (11) may be of elastic or flexible material. Furthermore, in an embodiment, the cap (7) may be defined with the cylindrical portion (11) and the conical portion (12) to accommodate the optical probes (3) extending from the head (8) of the optical device (1).

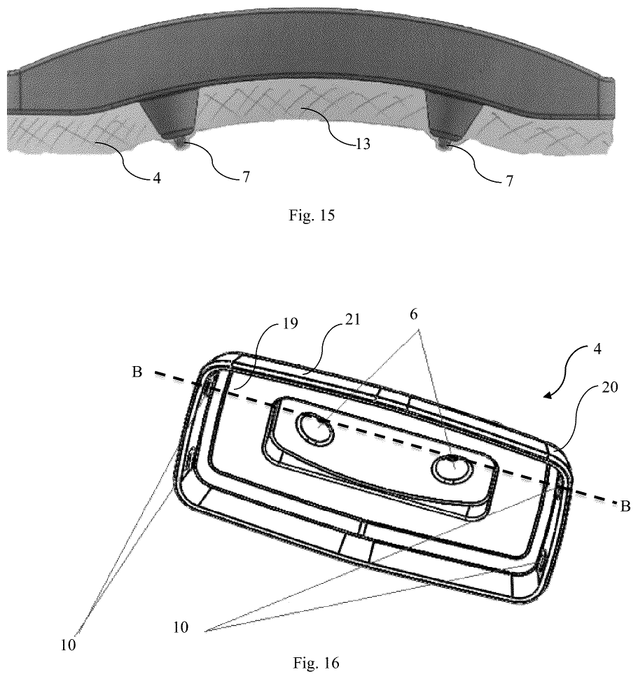

FIG. 14 illustrates an embodiment, depicting an uncompressed stage of the head (8) and the cover (4) where the head (8) is not in contact with the surface of the subject and FIG. 15 illustrates a compressed stage of the head (8) and the cover (4) where the head (8) is pressed against the surface of the subject. The cap (7) with at least a portion defined with the transparent material, covers the tip of the optical probes (3), and the remainder of the portions of the cover (4) are made of an opaque portion (13) [as seen in FIG. 14]. Further, FIG. 15 depicts an embodiment where the head (8) along with the cover (4) are pressed against the surface of the subject. Due to the compression, the tip of the optical probes (3) in the cap (7) aligns in line with the subject and compression of the cover (4) depends upon the curvature of the subject's surface. Even under maximum compressed stage, the cap (7), protrudes outwardly and covers the tip of the optical probe (3).

In an embodiment of the disclosure, the cover (4) may be made of hard and soft materials constructed in a way that it gives flexibility for the movement of the probe. The hardness may be varied depending on the thickness of the material at a particular point in the cover (4).

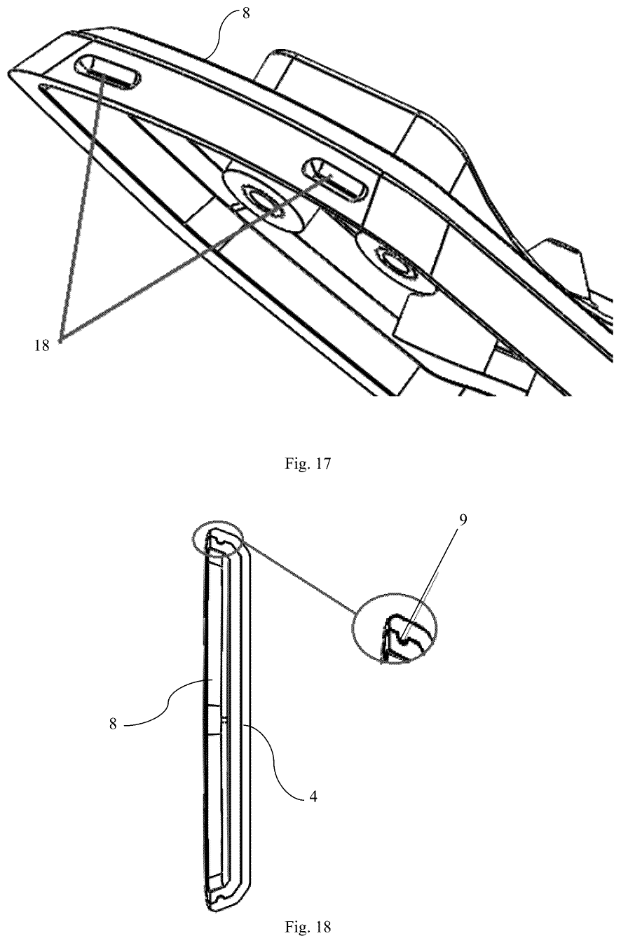

Now referring to FIG. 16 which illustrates a rear perspective view of the cover (4) and the FIG. 17 illustrates a perspective view of the head (8) of the optical device (1). The cover (4) and the head (8) may include a locking mechanism. The locking mechanism may be configured to lock the head (8) of the optical device (1) to secure the cover (4). The locking mechanism includes at least one locking tab (10) defined on the second surface (20) of the body (21) [as seen in FIG. 16]. Further, the locking mechanism may include a locking member (18) defined in the head (8) of the optical device (1) [as seen in FIG. 17]. The at least one locking tab (10) may be aligned and displaced relative to the at least one locking member (18) to lock the cover (4) with the head (8) of the optical device (1). The at least one locking tab (10) of the cover (4) may be configured to fit inside the locking member (18) of the head (8) and may be designed in such a way that the locking and unlocking can be achieved by hands of an operator. The at least one locking tab (10) and the locking member (18) enables a fitment (9) between the cover (4) and the head (8) of the optical device (1) [as seen in FIG. 18]. The locking mechanism enables the cover (4) to be removably connected to the head (8) and receive the optical probes (3). The cover (4) may be adapted to fit in a protective manner onto the optical probes (3) in a way that no air gaps exist between the optical probes (3) and the surface of the subject.

In an embodiment, the at least one locking tab (10) may be defined at predefined locations on the second surface (20) of the body (21). Further, the at least one locking tab (10) may be a continuous or elongated tab extending along the second surface (20) of the body (21). The locking member (18) may be including but not limited to a notch, a groove, a depression, a cavity (2) and any other provision to receive the at least one locking tab (10). The locking mechanism may be but not limited to a snap fit mechanism, a fastening mechanism, a friction hold mechanism and any other type capable of locking the cover (4) on the head (8) of the optical device (1).

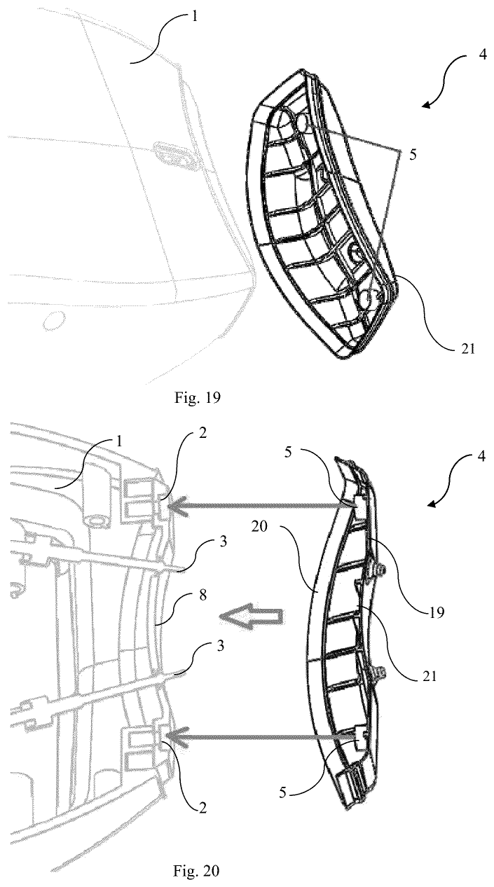

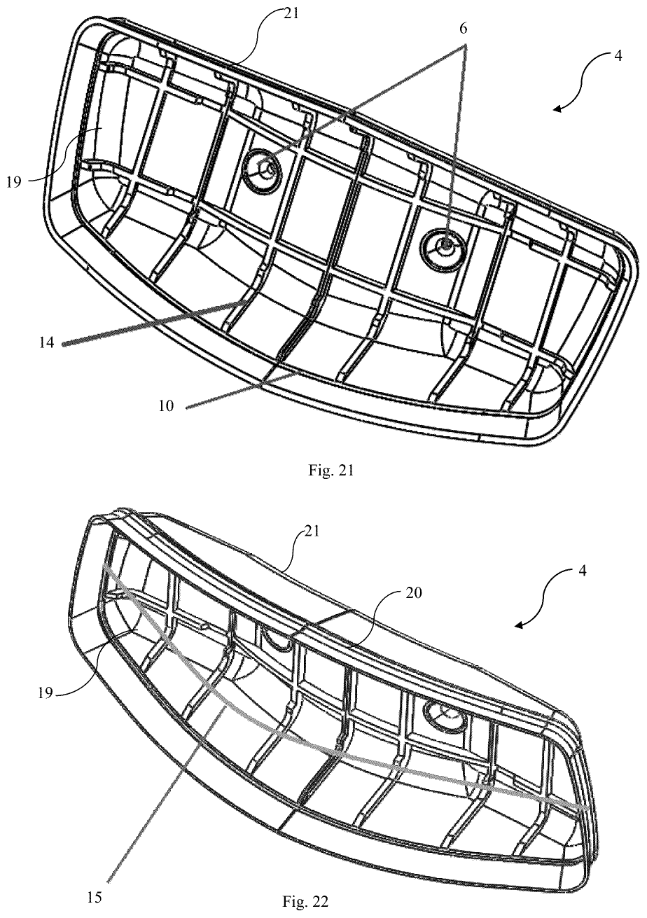

FIG. 19 illustrates a rear perspective view of the cover (4) and FIG. 20 illustrates a side cross sectional view of attaching the cover (4) with the head (8) of the optical device (1). At least one protrusion (5) may be defined on the first surface (19) of the body (21) on a face in a direction towards the head (8) of the optical device (1). The at least one protrusion (5) may be receivable in the cavity (2) defined in the head (8) of the optical device (1) upon locking the cover (4) on the head (8) of the optical device (1). The at least one protrusion (5) may be structured to activate the sensor system in the head (8) of the optical device (1) on being received in the cavity (2) to operate the optical device (1). The activation of the sensor system in the head (8) may allow the device to start the operation. The sensor system may be employed as a gateway to allow the optical device (1) to start operation upon the at least one protrusion (5) from the cover (4) being received in the cavity (2). Further, a microcontroller [not shown in Figs.] may be provided inside the optical device (1) and the microcontroller may read signal from the sensor system [not shown] to perform one or more functions as defined in the device firmware and mandates the usage of cover (4) to operate the device or may provide indication signals about the fitment (9) of the cover (4) on the head (8) of the optical device (1).

In an embodiment, the sensor system may be a pressure sensor, force sensor, stress gauge sensor, strain gauge sensor, touch sensor, flex sensor, proximity sensor, or any possible combination.

FIG. 21 illustrates a rear perspective view of the cover (4). As shown in FIG. 21, plurality of ribs (14) may be provided on the first surface (19) of the body (21) configured to contact the head (8) of the optical device (1). The plurality of ribs (14) define air pockets between the cover (4) and the head (8) of the optical device (1). The plurality of ribs (14) may provide a cushioning effect to the subject. Consequently, as the optical device (1) is pressed against the surface of the subject, the cover (4) may take the contour of the surface as the plurality of ribs (14) and the air pocket enable flexibility and compression to conform to a required shape. In an embodiment, the cover (4) may be configured to serve as a cushion in order to dissipate the force exerted through the optical device (1) over a larger surface area to minimize the pressure on the surface of application and minimize discomfort on the user or the subject.

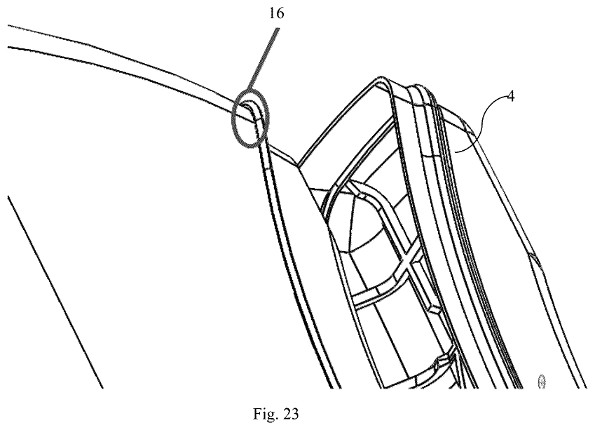

FIG. 22 illustrates a rear perspective view of the cover (4) with a seal (15). The seal (15) may be provided at a contact portion of the cover (4) and the head (8) of the optical device (1). In an embodiment, the seal (15) may be provisioned on the second surface (20) of the body (21). The seal (15) may be configured to torn-off or destroyed to attach the cover (4) on the head (8) of the optical device (1). The seal (15) may be made of a material capable of being ruptured by application of minimum force. The seal (15) ensures the cover (4) to be employed as a single usage cover (4) and avoid cross-contamination between different subjects, as a torn-off seal (15) is indicative of a used cover (4). In an embodiment, links may be provisioned on the first surface (19) of the cover (4) that need to be broken before positioning the cover (4) on the head (8) of the optical device (1). Further, the cover (4) may be configured to shrink after first locking and unlocking, thereby rendering the cover (4) non-usable for the second time. Also, a portion of the locking mechanism may be destroyed once the cover (4) is removed from the head (8) of the optical device (1) and preventing the cover (4) from locking with the head (8) of the optical device (1) for a second time.

FIG. 23 illustrates a rear perspective view of the cover (4) with a peeler (16) on the head (8) of the optical device (1). The peeler (16) may be configured to work as a swivel peeler i.e., while locking the cover (4), the peeler (16) may aid in gripping the locking member (18) with the at least one locking tab (10). Further, upon unlocking the cover (4), the peeler (16) may cut the cover (4) into two parts from the plane of the locking member (18) making the cover (4) non-usable for the second time. In an embodiment, the cover (4) may be configured to change color on being exposed to air, and thereby provide a suitable indication to the operator that the cover (4) is already used. In an embodiment, the cover (4) may be configured with an indication unit, for example a sticker, such that the sticker may have to be removed before attaching the cover (4) to the head (8) of the optical device (1).

In an embodiment, the material selection and molding procedure for manufacturing the cover (4) may be selected such that the cost of manufacturing may be reduced, and the cover (4) may be a low-cost disposable system.

In an embodiment, the cover (4) prevents misalignment of the optical probes (3). Further, two types of disposable covers (4) may be manufactured, one where the optical probes (3) are disposed together with the cover (4) after every operation or the other where only the cover (4) may be disposed after every operation. If the optical probes (3) are designed to be disposable, it increases the chance of misalignment at every usage, which may be mitigated due to the provision of the cover (4) around the optical probes (3).

In an embodiment, the material of the cover (4) does not induce any charge of its own. The cover (4) may act as an insulator between the surface of the subject and the optical device (1) which may be electronically charged or at a higher/lower temperature.

In an embodiment, the cover (4) prevents direct contact of the head (8) or the optical probes (3) with contaminants such as dirt, blood, sweat, salts, and the like, on the surface of the subject.

In an embodiment, the cover (4) may be water resistant, chemical corrosion resistant, non-toxic, acid proof, alkali proof, oil proof, odorless, shock proof, fire resistant, and may have low thermal coefficient.

In an embodiment, the cover (4) may be connected and disconnected from the head (8) of the optical device (1) without the need of external tools.

In an embodiment, the cover (4) may be employed to cover other examination devices including but not limited to ultrasound devices, radiation devices and the like.

In an embodiment, the cover (4) tightly holds the optical probes (3) and at the same time may be flexible for accommodation and maneuvering of the optical probes (3). Further, the cap (7) in the cover (4) ensures air pockets are not formed between the optical probe (3) and the surface of the subject.

In an embodiment, the cover (4) enables mandatory disposal of the cover (4) upon single usage. Further, the cover may be made of a soft material to provide comfort to the end user.

It should be imperative that configuration of the cover covering or enclosing the head of the optical device and any other elements or components described in the above detailed description should not be considered as a limitation with respect to the figures. Rather, variation to such structural configuration of the elements or components should be considered within the scope of the detailed description.

### EQUIVALENTS

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

Referral Numerals:

| Reference Number | Description |
|---|---|
| 1 | Optical device |
| 2 | Cavity |
| 3 | Optical probe |
| 4 | Cover |
| 5 | Protrusion |
| 6 | Aperture |
| 7 | Cap |
| 8 | Head |
| 9 | Fitment |
| 10 | Locking tab |
| 11 | Cylindrical portion |
| 12 | Conical portion |
| 13 | Opaque portion |
| 14 | Ribs |
| 15 | Seal |
| 16 | Peeler |
| 17 | Connection portion |
| 18 | Locking member |
| 19 | First surface |
| 20 | Second surface |
| 21 | Body |
| 22 | Stepped portion |

1. A disposable cover for covering a head of an optical device and contacting a subject for examination, the cover comprising:
a body, defining a first surface and a second surface extending along a periphery of the first surface in a direction substantially vertical to the first surface, the body is removably connectable to the head through the first surface and the second surface, wherein one or more apertures are defined in the first surface; and
a flexible cap disposed in each of the one or more apertures of the body, wherein at least a portion of the flexible cap is made of a substantially transparent material, the flexible cap is structured to accommodate an optical probe extending from the head of the optical device, such that the optical probe extends and retract relative to the body based on position of the optical device relative to the subject.

2. The cover as claimed in claim 1, wherein the body is configured to selectively filter light being either incident or reflected from at least one of the surface of the subject and surrounding of the body onto the head of the optical device.

3. The cover as claimed in claim 1, wherein the body includes a stepped portion extending from the first surface, the stepped portion is defined with the one or more apertures to accommodate the optical probe.

4. The cover as claimed in claim 1, wherein the flexible cap is positioned relative to the first surface of the body based on selective accommodation of the optical probes in the one or more apertures.

5. The cover as claimed in claim 1, wherein the flexible cap is configured to extend away from the first surface on receiving the optical probe to contact the subject.

6. The cover as claimed in claim 5, wherein the flexible cap defines a curved profile, when viewed along a cross-section of the flexible cap and the body at a connection portion with the one or more apertures, on extension of the flexible cap away from the first surface.

7. The cover as claimed in claim 1, wherein the flexible cap is configured to retract and flush with the first surface of the body, on contacting the subject for examination.

8. The cover as claimed in claim 7, wherein the flexible cap defines a substantially triangular profile, when viewed along a cross-section of the flexible cap and the body at the connection portion with the one or more apertures, on retraction of the flexible cap to flush with the first surface.

9. The cover as claimed in claim 1, comprises at least one protrusion defined on the first surface of the body, the at least one protrusion is receivable in a cavity defined in the head of the optical device to operate the optical device.

10. The cover as claimed in claim 9, wherein the at least one protrusion is structured to activate a sensor system in the head of the optical device on being received in the cavity to operate the optical device.

11. The cover as claimed in claim 1, comprises a locking mechanism defined on at least one of the body and the head of the optical device wherein the locking mechanism is configured to lock the head of the optical device to secure the cover.

12. The cover as claimed in claim 1, wherein the locking mechanism comprises:
at least one locking tab defined on the second surface of the body;
a locking member defined in the head of the optical device, wherein the at least one locking tab is aligned and displaceable relative to the at least one locking member to lock the cover with the head of the optical device.

13. The cover as claimed in claim 1, wherein the first surface of the cover is provisioned with plurality of ribs defining air pockets between the cover and the head of the optical device.

14. The cover as claimed in claim 1, wherein the second surface of the body is provisioned with a seal, configured to be torn-off to attach the cover on the head of the optical device.

15. The cover as claimed in claim 1, wherein the first surface and the second surface are defined with a profile complementary to a profile of the head of the optical device.
